(19) United States
(12) Patent Application Publication
(10) Pub. No.: US 2024/0375059 A1
(43) Pub. Date: Nov. 14, 2024

(54) METAL-ORGANIC FRAMEWORK (MOF)-801@CHITOSAN (CS) PERVAPORATION MEMBRANE AND USE THEREOF IN METHANOL/DIMETHYL CARBONATE SEPARATION

(71) Applicant: NANJING TECH UNIVERSITY, Nanjing (CN)

(72) Inventors: Gongping LIU, Nanjing (CN); Wanqin JIN, Nanjing (CN); Guozhen LIU, Nanjing (CN); Zhenggang WANG, Nanjing (CN); Xufang FU, Nanjing (CN)

(73) Assignee: NANJING TECH UNIVERSITY, Nanjing (CN)

(21) Appl. No.: 18/657,820

(22) Filed: May 8, 2024

(30) Foreign Application Priority Data
May 8, 2023 (CN) 202310513621.9

(51) Int. Cl. B01D 67/00; B01D 61/36; B01D 69/10; B01D 69/14; B01D 71/08; C07C 29/76; C07C 68/08

(52) U.S. Cl. CPC B01D 67/00793; B01D 61/362; B01D 69/105; B01D 69/107; B01D 69/148; B01D 71/08; C07C 29/76; C07C 68/08; B01D 2323/081; B01D 2323/12; B01D 2323/30; B01D 2323/60

(57) ABSTRACT

A preparation method of a metal-organic framework (MOF)-801@chitosan (CS) mixed matrix pervaporation membrane for separating methanol and dimethyl carbonate is provided. Chitosan (CS) is soluble in an acid and the synthesis of metal-organic framework (MOF)-801 requires an acid, an MOF-801@CS mixed matrix pervaporation membrane is creatively prepared in-situ. Because MOF-801 allows preferential adsorption for methanol and provides an additional transmission channel, a pore size-sieving role can be played, which improves the separation performance of the membrane for an organic azeotropic system of methanol/dimethyl carbonate (DMC). Compared with physical doping, the in-situ preparation method improves the dispersion uniformity of particles and the roughness of a surface of a membrane. Moreover, the membrane exhibits excellent swelling resistance and excellent structural stability in an organic methanol/DMC system.

### CROSS REFERENCE TO THE RELATED APPLICATIONS

This application is based upon and claims priority to Chinese Patent Application No. 202310513621.9, filed on May 8, 2023, the entire contents of which are incorporated herein by reference.

### TECHNICAL FIELD

The present disclosure relates to a membrane material, and specifically relates to a mixed matrix pervaporation membrane and a use thereof in methanol/dimethyl carbonate (DMC) separation.

### BACKGROUND

DMC is a colorless transparent liquid, and is widely used in pesticides, pharmaceuticals, polymer synthesis, fuel additives, and solvents. Currently, industrial production methods of DMC mainly include a liquid-phase methanol oxidative carbonylation method, a gas-phase methanol oxidative carbonylation method, and a transesterification method. However, a crude product initially obtained by each of the above methods is a mixture of DMC and methanol, which is a binary azeotropic mixture of methanol and DMC. The binary azeotropic mixture can hardly be separated by an ordinary separation method, and requires a special separation method to obtain pure DMC. Compared with the traditional separation methods (low-temperature crystallization, adsorption, azeotropic distillation, extractive distillation, and pressure-driven distillation), the membrane separation technology has advantages such as convenient operations, compact devices, safe working environments, and energy efficiency when used for the separation of a methanol/DMC mixture. Most importantly, the membrane separation technology can upset an azeotropic equilibrium between methanol and DMC.

In recent years, in order to overcome a “trade-off” effect between permeability and selectivity for the traditional polymer membranes and limitations such as high preparation cost and inherent brittleness of inorganic membranes, mixed matrix membranes have been provided to allow the optimization and complementation of membrane preparation processes and membrane performance. A filler is a core of a mixed matrix membrane, and thus the selection of the filler is crucial. Metal-organic frameworks (MOFs) are produced through the coordination of organic ligands with metal ions or clusters, and have both inorganic and organic properties. MOFs have better compatibility with polymers than the traditional inorganic fillers. Although there are many types of MOFs, most of MOFs have poor water stability and are difficult to exhibit a prominent separation effect during a liquid separation process. Carboxylate-based Zr-MOFs are produced by linking Zr metal clusters to ligands, most of the carboxylate-based Zr-MOFs have high coordination numbers and relatively-excellent hydrothermal temperatures. As a typical representative of Zr-MOF, MOF-801 (Zr₆O₄(OH)₄(fumarate)₆) is composed of a Zr ion, a hydroxide, and a fumarate, and has very prominent thermal stability and water stability. A three-dimensional framework of MOF-801 consists of Zr₆O₄(OH)₄ secondary building units (SBUs) and organic linkers of fumaric acid. Each Zr₆O₄(OH)₄ SBU includes 6 crystallographically-equivalent zirconium ions and coordinates with 12 fumaric acid linkers to form interconnected tetrahedra and octahedral cavities with 7.4 Å theoretical triangular windows. In this space group, two crystallographically-independent tetrahedral cavities have slightly different sizes and have diameters of 5.6 Å and 4.8 Å, respectively, which are between the kinetic diameter of 3.8 Å for methanol and the kinetic diameter of 6.0 Å for DMC and thus allows a prominent size-sieving capacity. Currently, mixed matrix membranes are mostly prepared by physical doping, which faces problems such as complicated membrane preparation process, large time consumption, and poor particle dispersion. The applicants have found that chitosan (CS) is insoluble in water and most organic solvents, but soluble in dilute acids such as formic acid and acetic acid, and the synthesis of MOF-801 requires the presence of an acid, which makes it possible to prepare an MOF-801/CS mixed matrix membrane through in-situ synthesis.

### SUMMARY

In view of the above problems, the present disclosure provides a method for in-situ preparation of a mixed matrix membrane, where MOF-801 with excellent hydrothermal stability and an appropriate pore size is used as a filler phase to prepare a pervaporation membrane for efficient separation of a methanol/DMC system.

The present disclosure provides an MOF-801@CS pervaporation membrane, where the MOF-801@CS pervaporation membrane includes a support and a mixed matrix layer for pervaporation, the mixed matrix layer includes a film-forming CS matrix and MOF-801 doped in the film-forming CS matrix, and a loading of the MOF-801 in the mixed matrix layer is 1 wt % to 15 wt %.

Preferably, an MOF-801@CS casting solution for preparing the mixed matrix layer is prepared in-situ by mixing ZrOCl₂, fumaric acid, and CS.

The present disclosure also provides a preparation method of the MOF-801@CS pervaporation membrane, including the following steps:
S1, adding appropriate amounts of a fumaric acid ligand and ZrOCl₂·8H₂O to an acetic acid aqueous solution in which the CS is dissolved to obtain a mixture, allowing the mixture to undergo a reaction in a water bath at a temperature for a period of time to obtain a first casting solution, taking the first casting solution out, and thoroughly stirring the first casting solution at room temperature to obtain a second casting solution;
S2, filtering the second casting solution to remove undissolved residues and impurities to obtain a third casting solution, and allowing the third casting solution to stand for bubble removal to obtain a fourth casting solution; and
S3, coating the fourth casting solution on the support to obtain a first membrane, drying the first membrane at room temperature to obtain a second membrane, soaking the second membrane in a sulfuric acid aqueous solution for crosslinking to obtain a third membrane, taking the third membrane out, and drying the third membrane at room temperature.

Preferably, in the S1, in the acetic acid aqueous solution, a concentration of the CS is 1 wt % to 5 wt % and a concentration of acetic acid is 1 wt % to 5 wt %; a molar ratio of the fumaric acid ligand to the ZrOCl₂·8H₂O is 1:(0.9-1.1); and a concentration of the fumaric acid ligand in the acetic acid aqueous solution is 0.04 wt % to 0.5 wt %.

Preferably, in the S1, the temperature for the reaction in the water bath is 40° C. to 80° C.

Preferably, in the S2, the filtering is conducted with a nylon mesh.

Preferably, the support used in the S3 is one or more selected from a group consisting of polyacrylonitrile (PAN), polysulfone, polyethersulfone, and polyvinylidene fluoride, the support has a pore size of 10 nm to 20 nm, and the support is soaked in water to remove impurities on a surface of the support.

Preferably, in the S3, a concentration of the sulfuric acid aqueous solution is 1 mmol·L⁻¹ to 3 mmol·L⁻¹.

Preferably, the crosslinking in the S3 is conducted for 12 h to 48 h.

The present disclosure also provides a use of the MOF-801@CS pervaporation membrane according to claim 1 in separation of methanol and DMC, where the methanol penetrates through the MOF-801@CS pervaporation membrane, and the DMC is retained.

Compared with the prior art, in the present disclosure, based on the characteristics that CS is soluble in an acid and the synthesis of MOF-801 requires an acid, an MOF-801@CS mixed matrix membrane is creatively prepared in-situ. Because MOF-801 allows preferential adsorption for methanol and provides an additional transmission channel, a pore size-sieving role can be played, which improves the separation performance of the membrane for an organic azeotropic system of methanol/DMC. Compared with physical doping, the in-situ preparation method improves the dispersion uniformity of particles and the roughness of a surface of a membrane. Moreover, the membrane exhibits excellent swelling resistance and excellent structural stability in an organic methanol/DMC system.

### BRIEF DESCRIPTION OF THE DRAWINGS

FIG. 1A shows a scanning electron microscopy (SEM) image of surface of a PAN support.

FIG. 1B shows a SEM image of cross section of a PAN support.

FIG. 1C shows a SEM image of surface of a pure membrane.

FIG. 1D shows a SEM image of cross section of a pure membrane.

FIG. 1E shows a SEM image of surface of a mixed matrix membrane with 4.3% doping amount.

FIG. 1F shows a SEM image of cross section of a mixed matrix membrane with 4.3% doping amount.

FIG. 1G shows a SEM image of surface of a mixed matrix membrane with 7.6% doping amount.

FIG. 1H shows a SEM image of cross section of a mixed matrix membrane with 7.6% doping amount.

FIG. 1I shows a SEM image of surface of a mixed matrix membrane with 13.3% doping amount.

FIG. 1J shows a SEM image of cross section of a mixed matrix membrane with 13.3% doping amount.

FIGS. 2A-2B show atomic force microscopy (AFM) images of a pure membrane (FIG. 2A) and a mixed matrix membrane (FIG. 2B).

FIG. 3 shows elemental analysis results of a mixed matrix membrane by energy-dispersive X-ray spectroscopy (EDS) mapping.

FIGS. 4A-4C show dynamic mechanical analysis (DMA) results of a pure membrane (FIG. 4A) and a mixed matrix membrane (FIG. 4B) and tensile strength test results (FIG. 4C) of a pure membrane and a mixed matrix membrane.

FIG. 5 shows pervaporation properties of MOF-801@CS mixed matrix membranes with different loadings.

FIGS. 6A-6D show electron microscopy images of surfaces of a particle (FIG. 6A) and a membrane (FIG. 6C), an X-ray diffraction (XRD) pattern of a particle (FIG. 6B), and performance comparison of membranes produced through in-situ synthesis and physical doping with an MOF-801/CS loading of 7.6 wt % (feed temperature: 50° C., and feed concentration: 10 wt % methanol) (FIG. 6D).

FIG. 7 shows long-term stability test results of a 7.6 wt % MOF-801/CS mixed matrix membrane.

### DETAILED DESCRIPTION OF THE EMBODIMENTS

### Example 1

43.5 mg of a fumaric acid ligand and 121 mg of ZrOCl₂·8H₂O were added to a 2 wt % acetic acid aqueous solution (100 mL) in which 2.5 wt % of CS had been dissolved to obtain a mixture, and the mixture was allowed to undergo a reaction in a water bath at 60° C. for a period of time, taken out, placed on a stirring table, and thoroughly stirred overnight at room temperature to obtain a first casting solution. The first casting solution was filtered with a nylon mesh to remove undissolved residues and impurities to obtain a second casting solution, and the second casting solution was allowed to stand for a period of time to remove bubbles in the second casting solution to obtain a third casting solution. A support used in the experiment was porous PAN (average pore size: about 15 nm), and before use, the support should be soaked in deionized water for about 2 d to remove glycerin on a surface of the support and then fully dried. The third casting solution was spin-coated on the support to obtain a first membrane, and the first membrane was naturally dried indoors, then soaked in a 2 mmol·L⁻¹ sulfuric acid aqueous solution to allow crosslinking for 24 h, then taken out, and dried at room temperature to obtain an MOF-801@CS pervaporation membrane with a doping amount of 4.3 wt %.

### Example 2

130.5 mg of a fumaric acid ligand and 363 mg of ZrOCl₂·8H₂O were added to a 2 wt % acetic acid aqueous solution (100 mL) in which 2.5 wt % of CS had been dissolved to obtain a mixture, and the mixture was allowed to undergo a reaction in a water bath at 60° C. for a period of time, taken out, placed on a stirring table, and thoroughly stirred overnight at room temperature to obtain a first casting solution. The first casting solution was filtered with a nylon mesh to remove undissolved residues and impurities to obtain a second casting solution, and the second casting solution was allowed to stand for a period of time to remove bubbles in the second casting solution to obtain a third casting solution. A support used in the experiment was porous PAN (average pore size: about 15 nm), and before use, the support should be soaked in deionized water for about 2 d to remove glycerin on a surface of the support and then fully dried. The third casting solution was spin-coated on the support to obtain a first membrane, and the first membrane was naturally dried indoors, then soaked in a 2 mmol·L⁻¹ sulfuric acid aqueous solution to allow crosslinking for 24 h, then taken out, and dried at room temperature to obtain an MOF-801@CS pervaporation membrane with a doping amount of 7.6 wt %.

### Example 3

217.5 mg of a fumaric acid ligand and 605 mg of ZrOCl₂·8H₂O were added to a 2 wt % acetic acid aqueous solution (100 mL) in which 2.5 wt % of CS had been dissolved to obtain a mixture, and the mixture was allowed to undergo a reaction in a water bath at 60° C. for a period of time, taken out, placed on a stirring table, and thoroughly stirred overnight at room temperature to obtain a first casting solution. The first casting solution was filtered with a nylon mesh to remove undissolved residues and impurities to obtain a second casting solution, and the second casting solution was allowed to stand for a period of time to remove bubbles in the second casting solution to obtain a third casting solution. A support used in the experiment was porous PAN (average pore size: about 15 nm), and before use, the support should be soaked in deionized water for about 2 d to remove glycerin on a surface of the support and then fully dried. The third casting solution was spin-coated on the support to obtain a first membrane, and the first membrane was naturally dried indoors, then soaked in a 2 mmol·L⁻¹ sulfuric acid aqueous solution to allow crosslinking for 24 h, then taken out, and dried at room temperature to obtain an MOF-801@CS pervaporation membrane with a doping amount of 13.3 wt %.

### Example 4

304.5 mg of a fumaric acid ligand and 847 mg of ZrOCl₂·8H₂O were added to a 2 wt % acetic acid aqueous solution (100 mL) in which 2.5 wt % of CS had been dissolved to obtain a mixture, and the mixture was allowed to undergo a reaction in a water bath at 60° C. for a period of time, taken out, placed on a stirring table, and thoroughly stirred overnight at room temperature to obtain a first casting solution. The first casting solution was filtered with a nylon mesh to remove undissolved residues and impurities to obtain a second casting solution, and the second casting solution was allowed to stand for a period of time to remove bubbles in the second casting solution to obtain a third casting solution. A support used in the experiment was porous PAN (average pore size: about 15 nm), and before use, the support should be soaked in deionized water for about 2 d to remove glycerin on a surface of the support and then fully dried. The third casting solution was spin-coated on the support to obtain a first membrane, and the first membrane was naturally dried indoors, then soaked in a 2 mmol·L⁻¹ sulfuric acid aqueous solution to allow crosslinking for 24 h, then taken out, and dried at room temperature to obtain an MOF-801@CS pervaporation membrane with a doping amount of 17.8 wt %.

### Characterization Results

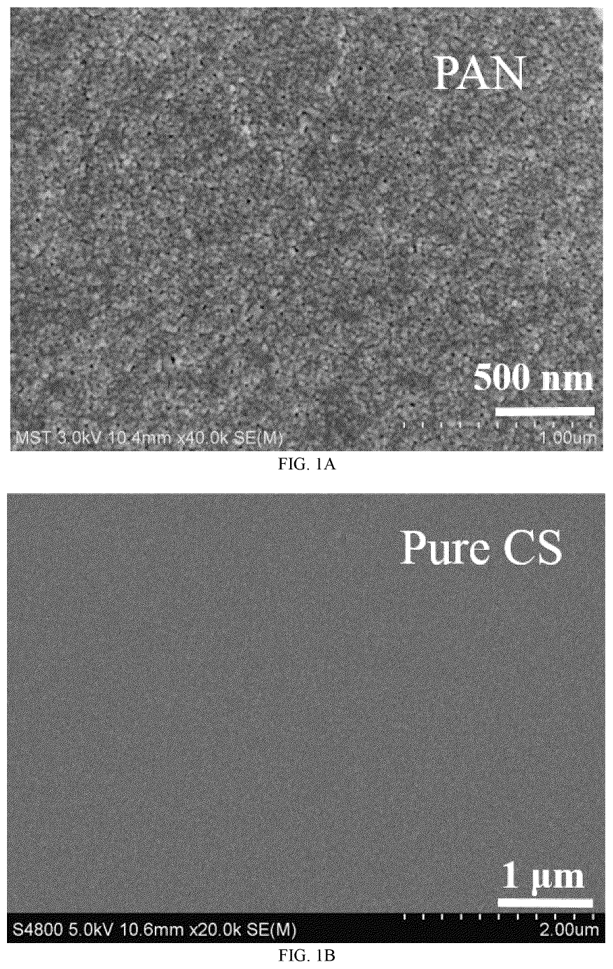

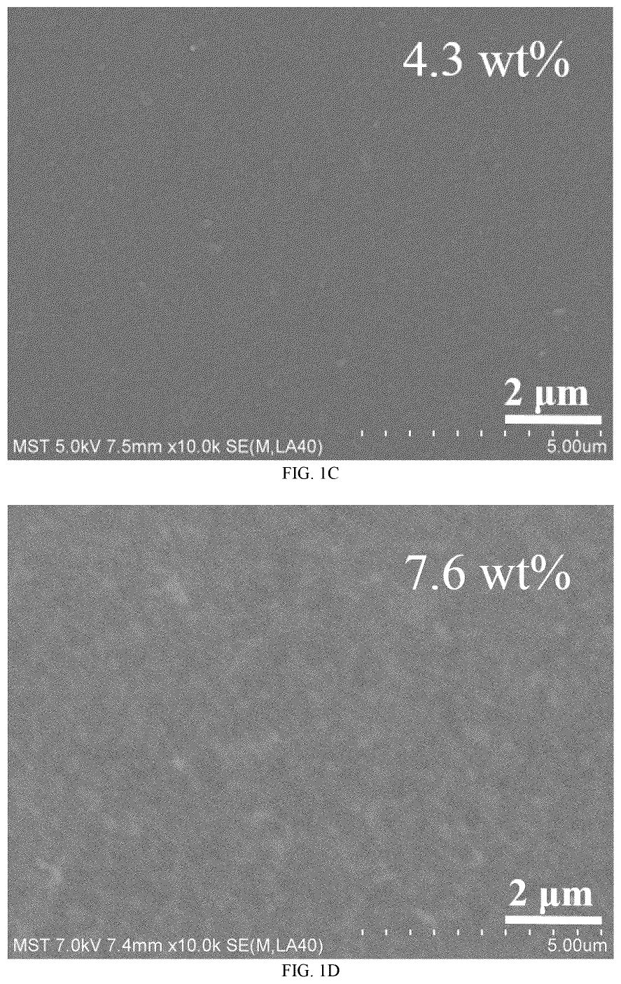

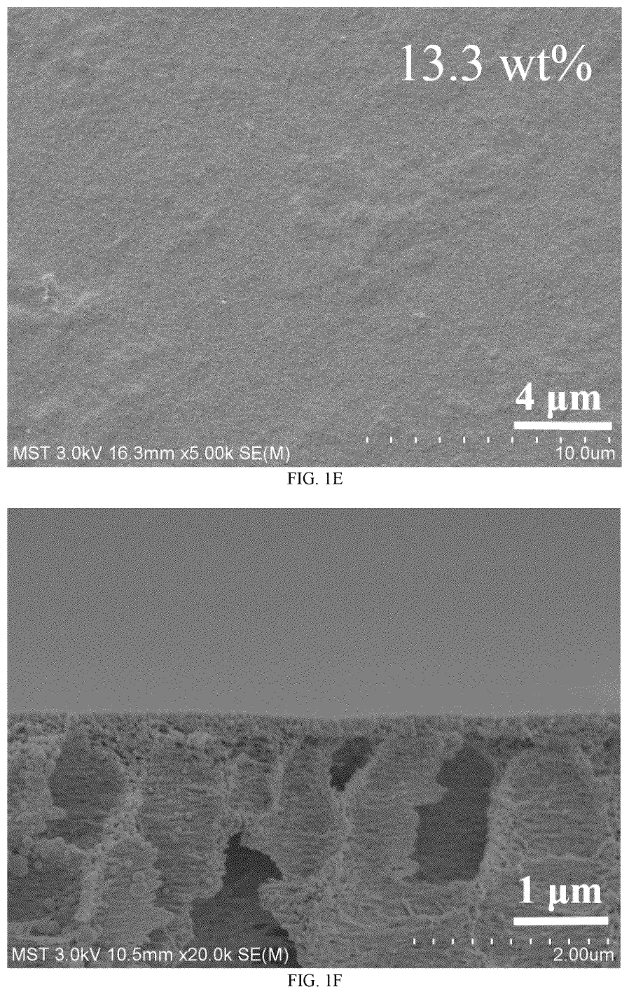

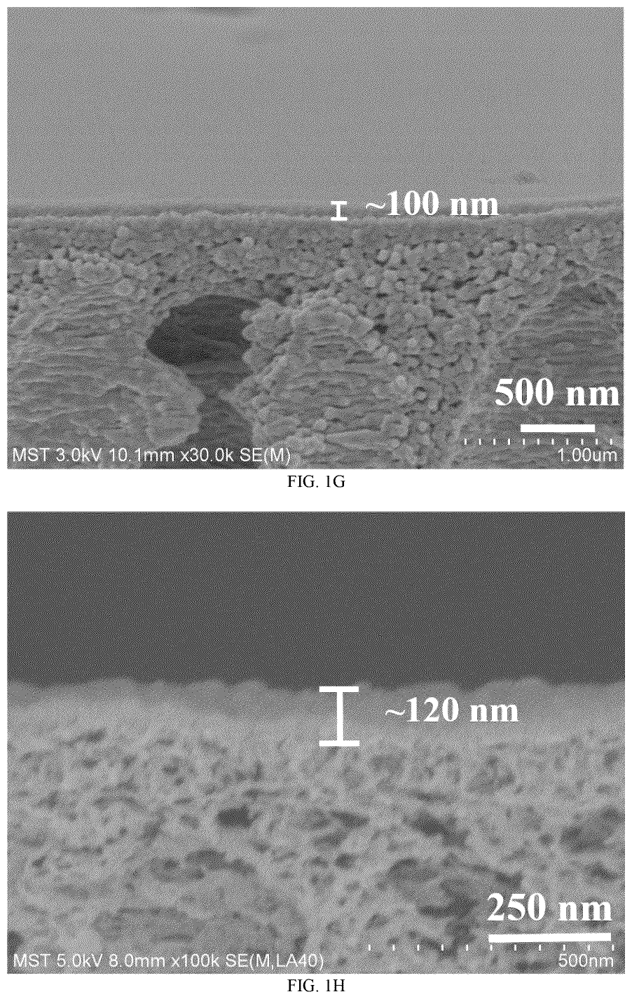

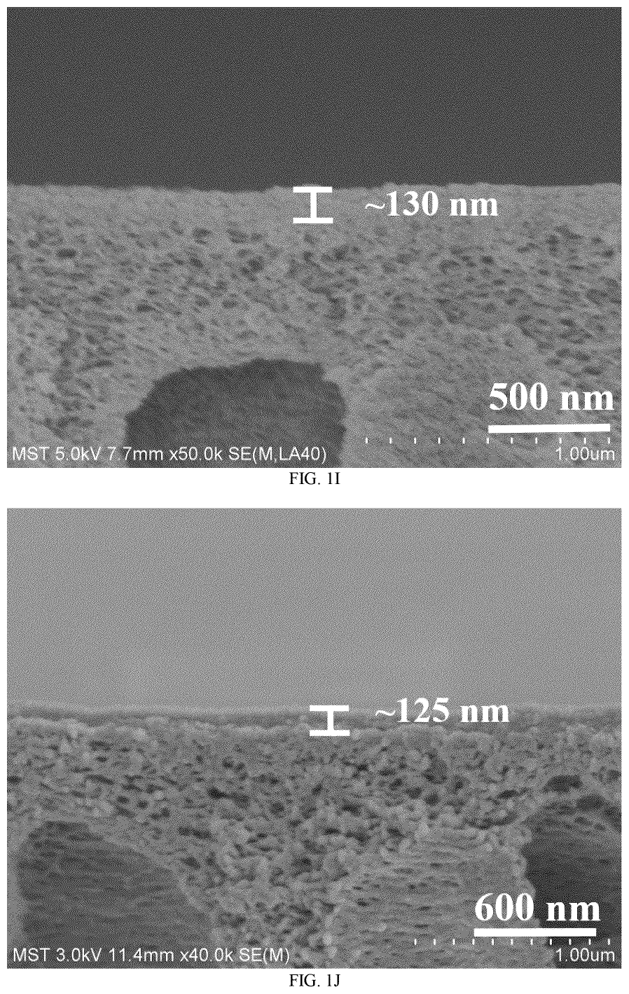

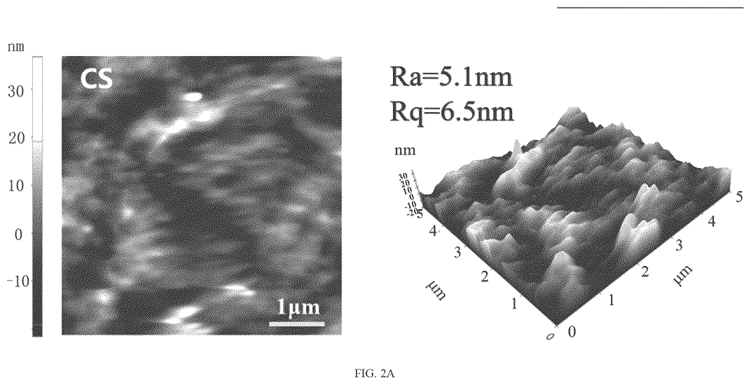

Morphologies of surfaces and cross sections of the PAN support and the mixed matrix membranes with different doping amounts were characterized by SEM, and results are shown in FIG. 1. It can be seen from this figure that there are many small pores with a diameter of about 15 nm on a surface of the PAN support. When a casting solution is coated on the support at a low doping amount, a resulting membrane has a smooth surface. With the increase of a doping amount, particles began to agglomerate on a surface of the membrane, but there is no obvious defect as a whole. When a doping amount is 13.3 wt %, the agglomeration of particles on the surface of the membrane is obvious. It can be seen from cross-section images that the prepared membranes all are relatively thin, and have a thickness of 100 nm to 200 nm.

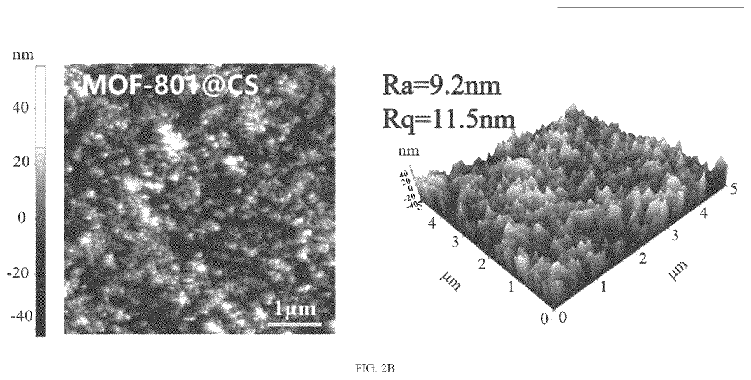

Then, morphologies of a pure membrane and a mixed matrix membrane were further characterized by AFM, and AFM images are shown in FIGS. 2A-2B. The pure CS membrane has a smooth and defect-free surface morphology, and the mixed matrix membrane has a surface with uniform protrusions. Both the pure membrane and the mixed matrix membrane have a relatively-low roughness. Compared with the pure CS membrane, a roughness of the mixed matrix membrane is improved to some extent, and Rq is increased from 6.5 nm to 11.5 nm. The surface roughness of the membrane is related to the surface wettability of the membrane, and also affects the separation performance of the membrane. The improvement of the roughness of the surface of the membrane means that a contact area of the surface of the membrane increases, and the increased contact area can increase an amount of a solvent contacting the surface of the membrane to affect the performance of the membrane.

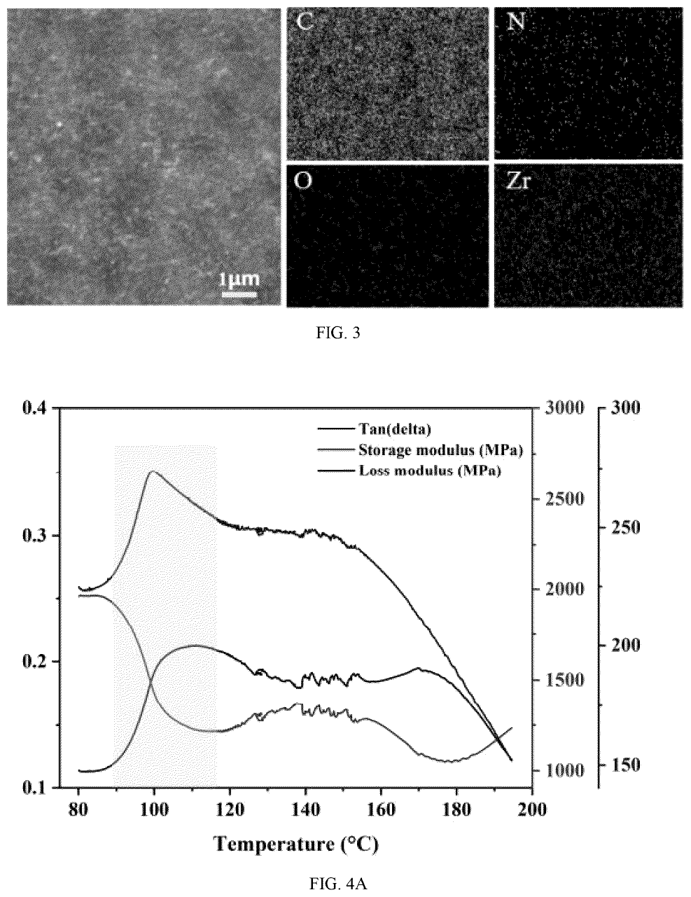

A mixed matrix membrane was subjected to elemental analysis by EDS, and results are shown in FIG. 3. In this figure, green, white, red, and blue dots represent carbon, nitrogen, oxygen, and zirconium elements, respectively. It can be seen from the elemental distribution maps that MOF-801 is successfully doped and doped particles are uniformly distributed in the membrane.

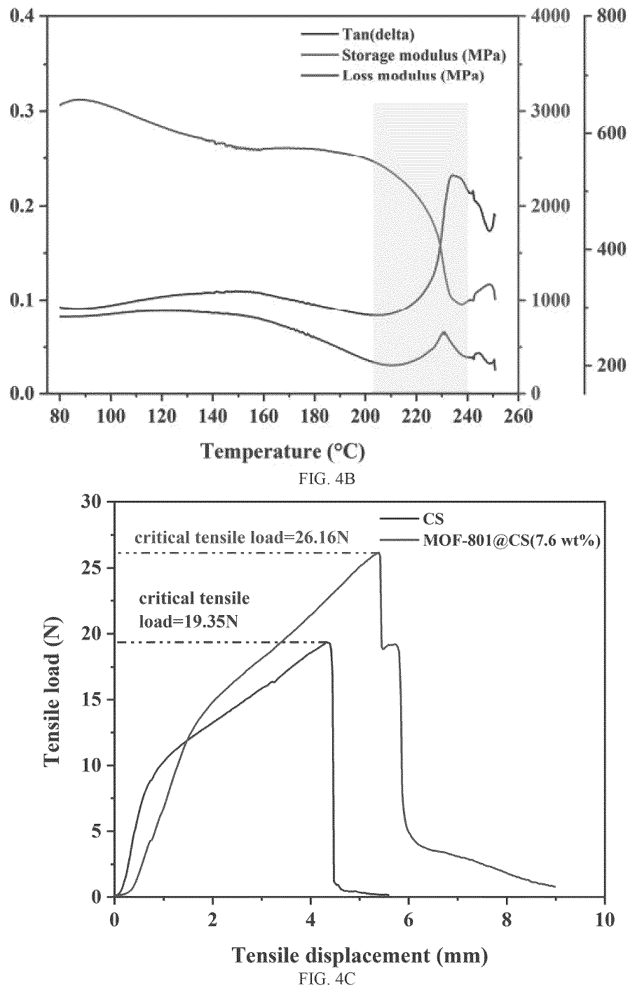

FIGS. 4A-4C show mechanical strength analysis results of membranes. During a DMA test, a test sample was heated according to a program, and an oscillation force was applied to allow a periodic oscillation, to obtain indexes of a material such as a dynamic storage modulus, a loss modulus, and a loss tangent angle. It can be seen from FIG. 4A and FIG. 4B that an inflection point of a storage modulus and a peak value of a loss tangent angle (tan δ) of the mixed matrix membrane both shift to a high temperature compared with the pure membrane, indicating that a glass transition temperature of the mixed matrix membrane is higher than a glass transition temperature of the pure membrane, and is improved from about 100° C. to about 220° C. The improvement of the glass transition temperature of the mixed matrix membrane means that the membrane is not prone to deformation, and can still retain a high strength and rigidity at a high temperature. It indicates that, after the particle doping, a polymer chain has enhanced rigidity, increased stability, weakened mobility, and improved mechanical performance, and requires an increased temperature to change from a glass state to a high-elastic state. In addition, a change of rigidity of the polymer chain can be seen according to changes of the storage modulus and the loss modulus. It is generally believed that the changes of the storage modulus and the loss modulus reflect the crystallinity and rigidity of a material. It can be seen from this figure that the storage modulus of the mixed matrix membrane is increased, and the loss modulus of the mixed matrix membrane is reduced, indicating that the higher the crystallinity, the stronger the rigidity and the better the mechanical performance. In combination with a thermogravimetric analysis curve, it can be known that a fluctuation of a DMA curve of the CS pure membrane at 130° C. to 150° C. may be caused by a change of mechanical performance due to thermal pyrolysis inside the CS pure membrane in this temperature range. Compared with the CS pure membrane, the mixed matrix membrane does not undergo any abnormality before the glass transition temperature due to its strong thermal stability. In order to intuitively verify the influence of particle doping on a mechanical strength of a membrane, the pure membrane and the mixed matrix membrane were cut into strip composite membranes of a same length and width and then tested by an electronic universal testing machine for a tensile strength, and results are shown in FIGS. 4A-4C. The mixed matrix membrane has stronger tensile resistance than the pure membrane, where the mixed matrix membrane has a critical tensile load of 26.16 N, and the pure membrane has a critical tensile load of 19.35 N. It indicates that the particle doping improves the rigidity and mechanical performance of the mixed matrix membrane, which is reflected in a difference in a tensile strength.

As shown in FIG. 5, the influence of an MOF-801 loading on the performance of an MOF-801@CS mixed matrix membrane was investigated, where a feed solution was a methanol/DMC solution with a concentration of 10 wt %, and a membrane test temperature was 50° C. Results show that the mixed matrix membranes (with loadings of 4.3 wt %, 7.6 wt %, and 13.3 wt %, respectively) all have a higher flux than the pure CS membrane, and a flux of the mixed matrix membrane increases gradually with the increase of an MOF-801 loading. In addition, when the MOF-801 loading increases from 0 to 7.6 wt %, a separation factor of the mixed matrix membrane tends to increase. This is because the doping of MOF-801 makes the mixed matrix membrane exhibit an improved adsorption capacity for methanol and thus have a specified pore size-sieving effect. During methanol permeation, due to the preferential adsorption of MOF-801, methanol preferentially passes through pores of the mixed matrix membrane, while a small amount of DMC is adsorbed in the pores and most of DMC passes through a polymer phase. DMC undergoes greater diffusion resistance than methanol during a permeation process, which also improves the diffusion selectivity of methanol. However, when the loading is too high, MOF-801 particles tend to agglomerate and cannot bind well to the polymer, such that microdefects occur at an interface between MOF-801 and the polymer matrix, which increases a flux and decreases a separation factor. Overall, when the loading is 7.6 wt %, a total flux and a separation factor of the mixed matrix membrane are higher than a total flux and a separation factor of the pure CS membrane, which breaks through the “trade-off” limitation between a flux and a separation factor of the traditional polymer.

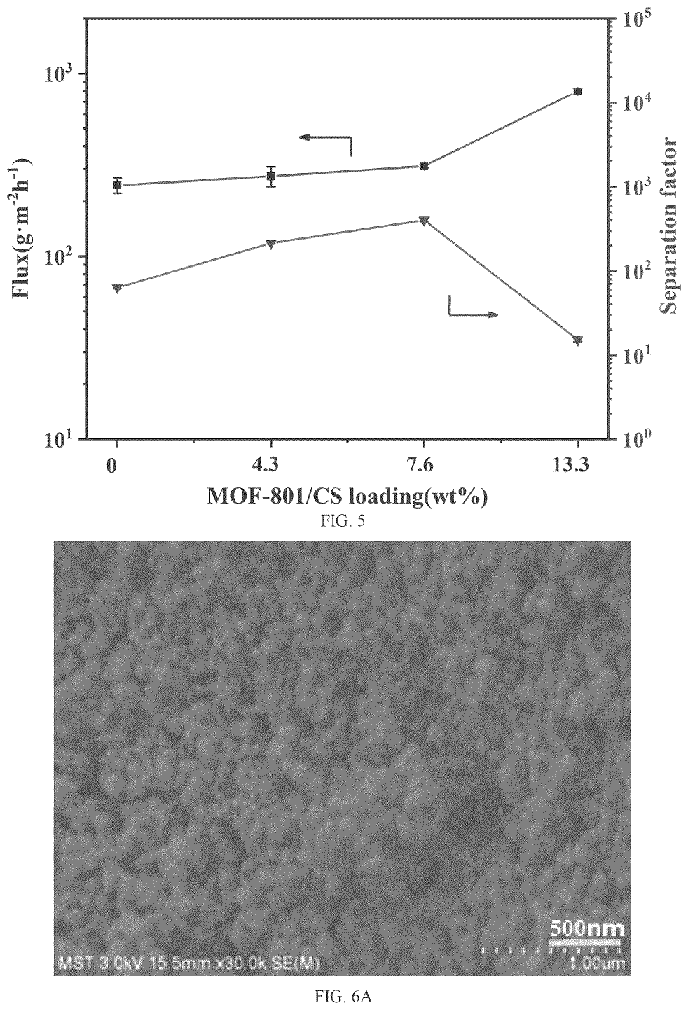

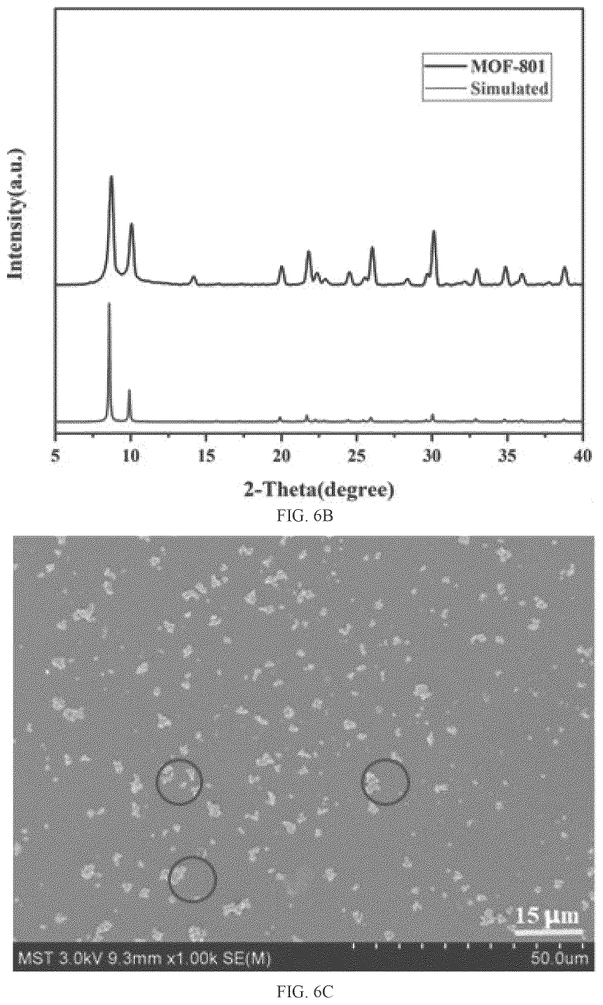

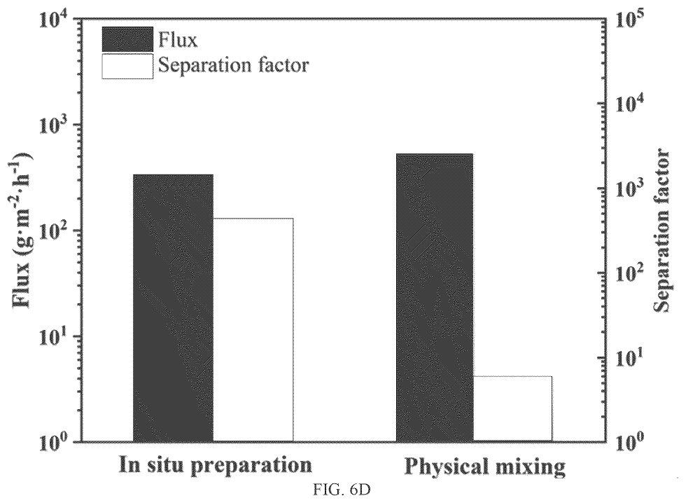

An MOF-801 mixed matrix membrane was prepared by physical mixing at the same doping amount. In FIGS. 6A-6D, FIG. 6A shows an electron microscope image of a particle first synthesized separately, and FIG. 6B shows an XRD pattern of the particle, which is consistent with a standard MOF-801 peak. It can be seen from FIG. 6C that the dispersion of particles on a surface of the membrane prepared by physical mixing is extremely uneven. The methanol/DMC separation performance of the mixed matrix membrane prepared through physical mixing was tested under the same conditions, and compared with the methanol/DMC separation performance of the mixed matrix membrane prepared through in-situ synthesis. Results show that the methanol/DMC separation performance of the mixed matrix membrane prepared through physical mixing is far lower than the methanol/DMC separation performance of the mixed matrix membrane prepared through in-situ synthesis, indicating that the in-situ synthesis has specified advantages, and the mixed matrix membrane prepared through in-situ synthesis has excellent particle dispersion.

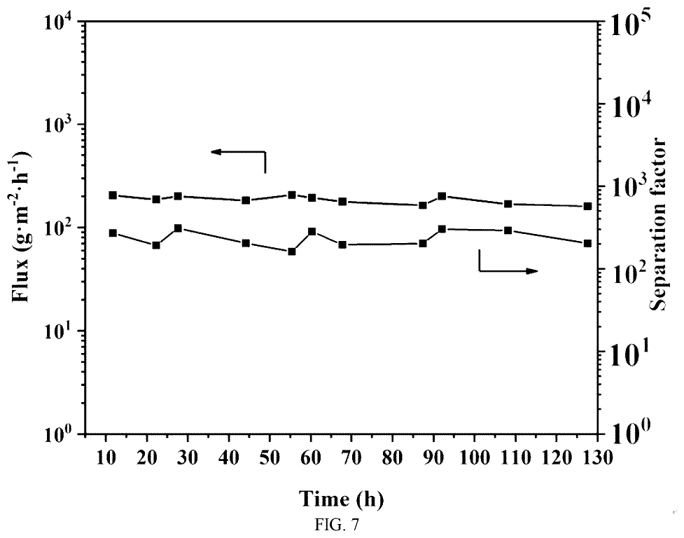

FIG. 7 shows long-term stability test results of a 7.6 wt % MOF-801@CS mixed matrix membrane, where a feed solution was 10 wt % methanol, and a test was conducted continuously for 130 h at 50° C. It can be seen from this figure that, during a 130 h long-term stability test, the performance of the mixed matrix membrane fluctuates slightly, but is generally stable, a flux of the mixed matrix membrane maintains at about 300 g·m⁻²·h⁻¹, and a separation factor of the mixed matrix membrane also maintains at about 300, indicating that the prepared MOF-801/CS mixed matrix membrane exhibits excellent swelling resistance and excellent structural stability in an organic methanol/DMC system.

When MOF-801 with a pore size-sieving effect for a methanol/DMC system is introduced through in-situ synthesis, a prepared MOF-801/CS mixed matrix membrane exhibits excellent separation performance with a separation factor as high as 403.

What is claimed is:

1. A preparation method of a metal-organic framework (MOF)-801@chitosan (CS) mixed matrix pervaporation membrane for separating methanol and dimethyl carbonate, wherein the MOF-801@CS mixed matrix pervaporation membrane comprises a support and a mixed matrix layer for a pervaporation, the mixed matrix layer comprises a film-forming chitosan matrix and MOF-801 doped in the film-forming chitosan matrix, and a loading of the MOF-801 in the mixed matrix layer is 7.6 wt % to 15 wt %; an MOF-801@CS casting solution for the mixed matrix layer is prepared in-situ by mixing ZrOCl₂, fumaric acid, and chitosan; and the preparation method comprises the following steps:
S1: adding appropriate amounts of a fumaric acid ligand and ZrOCl₂·8H₂O to an acetic acid aqueous solution dissolved with the chitosan to obtain a mixture, allowing the mixture to undergo a reaction in a water bath at a temperature for a period of time to obtain a first casting solution, taking the first casting solution out, and thoroughly stirring the first casting solution at room temperature to obtain a second casting solution;
S2: filtering the second casting solution to remove undissolved residues and impurities to obtain a third casting solution, and allowing the third casting solution to stand for a bubble removal to obtain a fourth casting solution; and
S3: coating the fourth casting solution on the support to obtain a first membrane, drying the first membrane at the room temperature to obtain a second membrane, soaking the second membrane in a sulfuric acid aqueous solution for a crosslinking to obtain a third membrane, taking the third membrane out, and drying the third membrane at the room temperature, wherein in the S1, in the acetic acid aqueous solution, a concentration of the chitosan is 1 wt % to 5 wt % and a concentration of acetic acid is 1 wt % to 5 wt %;
a molar ratio of the fumaric acid ligand to the ZrOCl₂·8H₂O is 1:(0.9-1.1); and a concentration of the fumaric acid ligand in the acetic acid aqueous solution is 0.04 wt % to 0.5 wt %.

2. The preparation method according to claim 1, wherein in the S1, the temperature for the reaction in the water bath is 40° C. to 80° C.

3. The preparation method according to claim 1, wherein in the S2, the second casting solution is filtered with a nylon mesh.

4. The preparation method according to claim 1, wherein the support used in the S3 is one or more selected from the group consisting of polyacrylonitrile, polysulfone, polyethersulfone, and polyvinylidene fluoride, the support has a pore size of 10 nm to 20 nm, and the support is soaked in water to remove impurities on a surface of the support.

5. The preparation method according to claim 1, wherein in the S3, a concentration of the sulfuric acid aqueous solution is 1 mmol·L⁻¹ to 3 mmol·L⁻¹.

6. The preparation method according to claim 1, wherein the crosslinking in the S3 is conducted for 12 h to 48 h.

7. A use of an MOF-801@CS mixed matrix pervaporation membrane prepared by the preparation method according to claim 1 in a separation of methanol and dimethyl carbonate, wherein the methanol penetrates through the MOF-801@CS mixed matrix pervaporation membrane, and the dimethyl carbonate is retained.
